(19) United States
(12) Patent Application Publication
(10) Pub. No.: US 2021/0343132 A1
(43) Pub. Date: Nov. 4, 2021

(54) METHOD AND SYSTEM OF PROVIDING AN EMERGENCY RESPONSE NOTIFICATION

(71) Applicant: Imran Bonser, Mirrabooka (AU)

(72) Inventor: Imran Bonser, Mirrabooka (AU)

(21) Appl. No.: 16/966,800

(22) PCT Filed: Jul. 5, 2018
(86) PCT No.: PCT/AU2018/050699

§ 371 Date: Jul. 31, 2020

(30) Foreign Application Priority Data
Jan. 8, 2018 (AU) 2018900045

(51) Int. Cl. G08B 21/04; G08B 25/00; A61B 5/00

(52) U.S. Cl. CPC G08B 21/0446; G08B 21/0453; A61B 5/0024; A61B 5/0022; G08B 25/006

(57) ABSTRACT

The present invention relates to a computer-implemented method of providing an automated emergency response alert for a patient, the method comprising: (a) sensing, via a sensor, patient information related to one more of activity, health, environment and location of the patient; (b) analyzing, with reference to predetermined emergency criteria, the patient information received from the sensor via a system in communication with the sensor; (c) selecting an emergency response alert based on the analysis of the patient information received from the sensor; and (d) issuing the selected emergency response alert via a communications network.

### TECHNICAL FIELD

The disclosure relates, generally, to a computer-implemented method and system of patient/user monitoring utilizing wearable sensor technology and, more particularly, to a computer-implemented method and system for automatically generating an emergency response notification for that patient/user. The disclosure has particular, but not necessarily exclusive, application to monitoring systems for the elderly and automatically generating emergency response notifications in response to measured changes concerning a user's health, activity, environment and/or location.

### BACKGROUND

Existing wearable devices, and specifically wearable sensors utilizing MEMS (Micro Electro Mechanical Systems), are commonplace in the personal health and fitness industries. More specifically, a portable computing device incorporating or controlling such sensors can be readily fashioned into a wearable device or accessory such as, for example, a watch, a pendant, a bracelet, a brooch, etc. It is common for such wearable accessories to be affixed to a limb of a user's body such as, for example, the user's wrist or ankle. Depending on the specific positioning of the wearable device, and the particular MEMS contained within that device, different characteristic information about the user (i.e. wearer of the device) can be gathered and presented to the user (if the wearable device incorporates a graphical interface, or communication link to an associated mobile device having a graphical interface e.g. a cell phone or tablet device).

There has been limited use of such wearable devices in systems for monitoring the health of elderly or infirm users or patients. However, these systems generally require the user/patient to have an associated mobile device (e.g. cell phone, tablet, or laptop computer), and/or access to a computer with a graphical interface, in order to view the data captured by the device. It is not generally not practical for elderly patients, or patients with prolonged illnesses, to efficiently access and operate such mobile devices in order to view and analyze the data captured by the wearable device. As an alternative, some wearable devices incorporate their own graphical interface which, due to the small size of such wearable devices, is also relatively small and difficult to view. Again, considering that the intended users of such devices are the elderly and infirm, it is simply not practical to have the user's data presented on such a small display on the device.

Of the above described systems and devices used for monitoring the health of elderly or infirm users or patients, some provide the functionality to upload patient data directly to a website, or even send such data to a clinician (by, for example, email or other electronic transmission means). However, there is no requirement upon (nor is there currently any mechanism to allow) the clinician to view or act upon the patient's data in real time such as, for example, in the event of an emergency event. Similarly, these systems provide no functionality to determine the likely urgency of a patient's emergency event (for example, whether a patient's family or friends can assist with addressing the ‘event’, whether the ‘event’ can be addressed by a clinician within a moderate (1-2 day timeframe), or whether immediate ambulance response is required).

In view of the above mentioned problems of the prior art, there is a need for an improved method and system for monitoring the health of a patient (or user) in real time. There is also a need for an improved method and system for generating automated emergency response notifications (to appropriate respondents) in the event that the patient is deemed to require assistance, and to allow those respondents to quickly and efficiently locate the patient in order to render assistance.

In this specification where a document, act or item of knowledge is referred to or discussed, this reference or discussion is not an admission that the document, act or item of knowledge or any combination thereof was at the priority date, publicly available, known to the public, part of the common general knowledge; or known to be relevant to an attempt to solve any problem with which this specification is concerned.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

The present disclosure relates to a computer-implemented method of providing an automated emergency response alert for a patient, the method comprising:

(a) sensing, via a sensor, patient information related to one more of activity, health, environment and location of the patient;

(b) analyzing, with reference to predetermined emergency criteria, the patient information from the sensor via a system in communication with the sensor;

(c) selecting an emergency response alert based on the analysis of the patient information received from the sensor; and

(d) issuing the selected emergency response alert via a communications network.

In the step of sensing, the sensor may be positioned against or in close proximity to the patient's skin. In a representative embodiment of the present disclosure, the sensor may be located within a wearable device worn by the patient. This wearable device may, for example, be fashioned in the form of a watch, a pendant, a bracelet, a brooch, etc., or any similar form that allows for capture of patient information from the patient.

The patient information, relative to the patient's activity, may include one or more of the patient's relative movement as a function of time, the patient's movement relative to the patient's predefined movement pattern; and the patient's movement relative to a monitoring hub located within the patient's dwelling. The patient's relative movement as a function of time may include, for example, the speed at which the patient is moving, as well as any rapid changes in the height of the sensor worn by the patient (which may be indicative of a fall by the patient).

The predetermined emergency criteria, relative to the patient's activity, may include one or more of a predetermined minimum patient movement frequency, a predetermined maximum delay between recorded patient movements, and a predetermined movement pattern for the patient. In an elderly patient, for example, while a high level of movement would not be expected, it would also be irregular if the patient did not exhibit any movement for a prolonged period of time. Similarly, the patient's movement patterns may, for example, specify (based on historical data) that a patient has a certain raised level of activity at particular times throughout the day (e.g. breakfast, lunch, dinner, and scheduled activity periods), and low levels of activity during the night. With regard to elderly patients, whose movements and activity levels tend to be quite routine, these movement patterns can be tracked and recorded to a fairly high level of accuracy (particularly for those elderly patients living in nursing homes).

The patient information, relative to the patient's health, may include one or more of the patient's heart rate, the patient's blood pressure, the patient's body temperature, the patient's blood sugar level; and the patient's electrocardiography.

The predetermined emergency criteria, relative to the patient's health, may include one or more of a predetermined minimum and/or maximum heart rate, a predetermined minimum and/or maximum blood pressure, a predetermined minimum and/or maximum body temperature, a predetermined minimum and/or maximum blood sugar level, and a predetermined range of expected electrocardiography readings. Such predetermined emergency criteria may be set with general parameters (e.g. parameters that would indicate abnormal health conditions for an ‘ordinary’ person) or may be customised based on the patient's own medical history (for example, in the case of a patient with known blood pressure issues, the system would allow for that patient's minimum and/or maximum blood pressure parameters in their predetermined emergency criteria to be modified to reflect that pre-existing condition).

The patient information, relative to the patient's environment, may include one or more of the ambient temperature of the patient's environment, and the air quality of the patient's environment.

The predetermined emergency criteria, relative to the patient's environment, may include one or more of a predetermined maximum ambient temperature, and a predetermined minimum air quality. For example, environmental conditions that could otherwise endanger the health of the patient (such as those caused by fire or heating system malfunction) could be mitigated by setting a maximum ambient temperature of say 50 degrees Celsius. Similarly, an air quality measure could be specified to create an alert when the quality of the air in the patient's immediate environment drops below a specified minimum (as may well occur in the case of a fire or smoky conditions, or where a gas leak has occurred).

The patient information, relative to the patient's location, may include one or more of the patient's GPS position, and the patient's location relative to a monitoring hub located within the patient's dwelling.

The predetermined emergency criteria, relative to the patient's location, may include one or more of a predetermined GPS position, and a predetermined maximum distance from the monitoring hub located within the patient's dwelling. It should be appreciated that the predetermined GPS position may also include a ‘geo-fenced’ region defined by GPS coordinates. For example, the ‘geo-fenced’ region may be established to identify the perimeter of a nursing home in which the patient is a resident. As a result, the patient's GPS position relative to the predetermined GPS position (i.e. the ‘geo-fenced’ region) would identify whether the patient is still present within the nursing home.

The step of selecting may further comprise determining, based on a comparison between the patient information and predetermined emergency criteria, whether an emergency event has occurred, and assigning, based on a comparison between the patient information and predetermined emergency criteria, an alert priority level to the emergency event.

The alert priority level may comprise one of the following: a low-range emergency response; a mid-range emergency response; or a critical emergency response. In a representative embodiment of the present disclosure, and by way of example, a low-range emergency response may be one that can be adequately addressed by a patient's friends or family (i.e. the emergency event is not life threatening and does not require an immediate response). Similarly, a mid-range emergency response may be one that the patient's clinician (or in the case of a patient in a nursing home, the nursing or medical staff of the nursing home) can adequately address (i.e. the emergency event is not life threatening and does not require an immediate response). Finally, and also by way of example, a critical emergency response may be one that requires immediate action by emergency medical professionals (such as, for example, an ambulance or emergency services).

The step of issuing may further comprise determining, based on the alert priority level assigned to the emergency event, at least one category of recipient for the emergency response alert, and issuing the selected emergency response alert via the communications network to the at least one category of recipient.

The at least one category of recipient may be selected from predefined recipient categories. These predefined recipient categories may comprise primary carer response category, non-critical medical response category, and critical medical response category. As with the categorisation of the alert priority levels, and in a representative embodiment of the present disclosure, the primary carer response category may include members of the patient's friends or family, the non-critical response category may include the patient's clinician (or in the case of a patient in a nursing home, the nursing or medical staff of the nursing home), and the critical medical response category may include emergency medical professionals (such as, for example, an ambulance or emergency services).

Software that when installed on a mobile communication device may cause the mobile communication device to perform the above method. An Application Programming Interface that when installed on a mobile communication device as part of a patient application may cause the mobile communication device to perform the above method.

The present disclosure also relates to a computer-implemented system of providing an automated emergency response alert for a patient, the system comprising:

a sensor for sensing patient information related to one more of activity, health, environment and location of the patient; and

a processing system configured to perform the above described method, wherein the processing system is a server processing system.

In a representative embodiment of the present disclosure, the sensor may be located within a wearable device worn by the patient. This wearable device may, for example, be fashioned in the form of a watch, a pendant, a bracelet, a brooch, etc., or any similar form that allows for capture of patient information from the patient. More specifically, the sensor may be positioned against or in close proximity to the patient's skin depending on the type(s) of patient information required.

The present disclosure also relates to a computer-implemented system of providing an automated emergency response alert for a patient, the system comprising:

a computer server accessible through a communications network, the computer server arranged to receive patient information through the communications network;

a processor, communicatively coupled to the computer server, to one or more means for graphical display of information, and to one or more means for receiving input, the processor being configured to:
(a) sense, via a sensor, patient information related to one more of activity, health, environment and location of the patient;
(b) analyze, with reference to predetermined emergency criteria, the patient information from the sensor via a system in communication with the sensor;
(c) select an emergency response alert based on the analysis of the patient information received from the sensor; and
(d) issue, via the communications network, the selected emergency response alert via a communications network.

In a representative embodiment of the present disclosure, the sensor may be located within a wearable device worn by the patient. This wearable device may, for example, be fashioned in the form of a watch, a pendant, a bracelet, a brooch, etc., or any similar form that allows for capture of patient information from the patient. More specifically, the sensor may be positioned against or in close proximity to the patient's skin depending on the type(s) of patient information required.

The patient information, relative to the patient's activity, may include one or more of the patient's relative movement as a function of time, the patient's movement relative to the patient's predefined movement pattern; and the patient's movement relative to a monitoring hub located within the patient's dwelling. The patient's relative movement as a function of time may include, for example, the speed at which the patient is moving, as well as any rapid changes in the height of the sensor worn by the patient (which may be indicative of a fall by the patient).

The predetermined emergency criteria, relative to the patient's activity, may include one or more of a predetermined minimum patient movement frequency, a predetermined maximum delay between recorded patient movements, and a predetermined movement pattern for the patient. In an elderly patient, for example, while a high level of movement would not be expected, it would also be irregular if the patient did not exhibit any movement for a prolonged period of time. Similarly, the patient's movement patterns may, for example, specify (based on historical data) that a patient has a certain raised level of activity at particular times throughout the day (e.g. breakfast, lunch, dinner, and scheduled activity periods), and low levels of activity during the night. With regard to elderly patients, whose movements and activity levels tend to be quite routine, these movement patterns can be tracked and recorded to a fairly high level of accuracy (particularly for those elderly patients living in nursing homes).

The patient information, relative to the patient's health, may include one or more of the patient's heart rate, the patient's blood pressure, the patient's body temperature, the patient's blood sugar level; and the patient's electrocardiography.

The predetermined emergency criteria, relative to the patient's health, may include one or more of a predetermined minimum and/or maximum heart rate, a predetermined minimum and/or maximum blood pressure, a predetermined minimum and/or maximum body temperature, a predetermined minimum and/or maximum blood sugar level, and a predetermined range of expected electrocardiography readings. Such predetermined emergency criteria may be set with general parameters (e.g. parameters that would indicate abnormal health conditions for an ‘ordinary’ person) or may be customised based on the patient's own medical history (for example, in the case of a patient with known blood pressure issues, the system would allow for that patient's minimum and/or maximum blood pressure parameters in their predetermined emergency criteria to be modified to reflect that pre-existing condition).

The patient information, relative to the patient's environment, may include one or more of the ambient temperature of the patient's environment, and the air quality of the patient's environment.

The predetermined emergency criteria, relative to the patient's environment, may include one or more of a predetermined maximum ambient temperature, and a predetermined minimum air quality. For example, environmental conditions that could otherwise endanger the health of the patient (such as those caused by fire or heating system malfunction) could be mitigated by setting a maximum ambient temperature of say 50 degrees Celsius. Similarly, an air quality measure could be specified to create an alert when the quality of the air in the patient's immediate environment drops below a specified minimum (as may well occur in the case of a fire or smoky conditions, or where a gas leak has occurred).

The patient information, relative to the patient's location, may include one or more of the patient's GPS position, and the patient's location relative to a monitoring hub located within the patient's dwelling.

The predetermined emergency criteria, relative to the patient's location, may include one or more of a predetermined GPS position, and a predetermined maximum distance from the monitoring hub located within the patient's dwelling. It should be appreciated that the predetermined GPS position may also include a ‘geo-fenced’ region defined by GPS coordinates. For example, the ‘geo-fenced’ region may be established to identify the perimeter of a nursing home in which the patient is a resident. As a result, the patient's GPS position relative to the predetermined GPS position (i.e. the ‘geo-fenced’ region) would identify whether the patient is still present within the nursing home.

The processor may be further configured to determine, based on a comparison between the patient information and predetermined emergency criteria, whether an emergency event has occurred, and assign, based on a comparison between the patient information and predetermined emergency criteria, an alert priority level to the emergency event.

The alert priority level may comprise one of the following: a low-range emergency response; a mid-range emergency response; or a critical emergency response. In a representative embodiment of the present disclosure, and by way of example, a low-range emergency response may be one that can be adequately addressed by a patient's friends or family (i.e. the emergency event is not life threatening and does not require an immediate response). Similarly, a mid-range emergency response may be one that the patient's clinician (or in the case of a patient in a nursing home, the nursing or medical staff of the nursing home) can adequately address (i.e. the emergency event is not life threatening and does not require an immediate response). Finally, and also by way of example, a critical emergency response may be one that requires immediate action by emergency medical professionals (such as, for example, an ambulance or emergency services).

The processor may be further configured to determine, based on the alert priority level assigned to the emergency event, at least one category of recipient for the emergency response alert, and issuing the selected emergency response alert via the communications network to the at least one category of recipient.

The at least one category of recipient may be selected from predefined recipient categories. These predefined recipient categories may comprise primary carer response category, non-critical medical response category, and critical medical response category. As with the categorisation of the alert priority levels, and in a representative embodiment of the present disclosure, the primary carer response category may include members of the patient's friends or family, the non-critical response category may include the patient's clinician (or in the case of a patient in a nursing home, the nursing or medical staff of the nursing home), and the critical medical response category may include emergency medical professionals (such as, for example, an ambulance or emergency services).

The present disclosure also relates to a computer-implemented method as performed by a mobile application installed on a mobile communication device to facilitate the provision of an automated emergency response alert for a patient, the method comprising:

(a) sensing, via a sensor, patient information related to one more of activity, health, environment and location of the patient;

(b) analyzing, with reference to predetermined emergency criteria, the patient information from the sensor via a system in communication with the sensor;

(c) selecting an emergency response alert based on the analysis of the patient information received from the sensor; and

(d) issuing, via a communications network, the selected emergency response alert.

The communication device may comprise a display device to facilitate interaction by the patient with the mobile application.

Software that when installed on a mobile communication device may cause the mobile communication device to perform the above method. An Application Programming Interface that when installed on a mobile communication device as part of a patient application may cause the mobile communication device to perform the above method.

The present disclosure also relates to a mobile communication device comprising:

a sensor, preferably located within a wearable device worn by the patient;

a program memory to store a patient application installed on the mobile communication device;

a data port to facilitate communication with an application server via a communications network; and

a processor to
(a) sense, via the sensor, patient information related to one more of activity, health, environment and location of the patient;
(b) analyze, with reference to predetermined emergency criteria, the patient information from the sensor via a system in communication with the sensor;
(c) select an emergency response alert based on the analysis of the patient information received from the sensor; and
(d) issue, via the communications network, the selected emergency response alert.

The mobile communication device may further comprise a display device and an input device to facilitate interaction by the patient with the patient application.

### BRIEF DESCRIPTION OF DRAWINGS

Embodiments of the present invention will now be described with reference to the accompanying drawings. These embodiments are given by way of illustration only and other embodiments of the invention are also possible. Consequently, the particularity of the accompanying drawings is not to be understood as superseding the generality of the preceding description. In the drawings:

FIG. 1 is a schematic block diagram illustrating a system of providing an automated emergency response alert for a patient in accordance with a representative embodiment of the present disclosure;

FIG. 2 is a schematic block diagram illustrating a web-based system of a system of providing an automated emergency response alert for a patient in accordance with an alternative embodiment of the present disclosure; and

FIG. 3 is a schematic block diagram illustrating a system of providing an automated emergency response alert for a patient in accordance with an alternative embodiment of the present disclosure.

### DESCRIPTION OF EMBODIMENTS

Representative embodiments of the present disclosure relate, generally, to a computer-implemented method and system of patient/user monitoring utilizing wearable sensor technology and, more particularly, to a computer-implemented method and system for automatically generating an emergency response notification for that patient/user. The disclosure has particular, but not necessarily exclusive, application to monitoring systems for the elderly and automatically generating emergency response notifications in response to measured changes concerning a user's health, activity, environment and/or location. However, it should be understood that the disclosure is not limited to this representative embodiment, and may be implemented in other environments such as, for example, potentially hazardous environments where users are required to work.

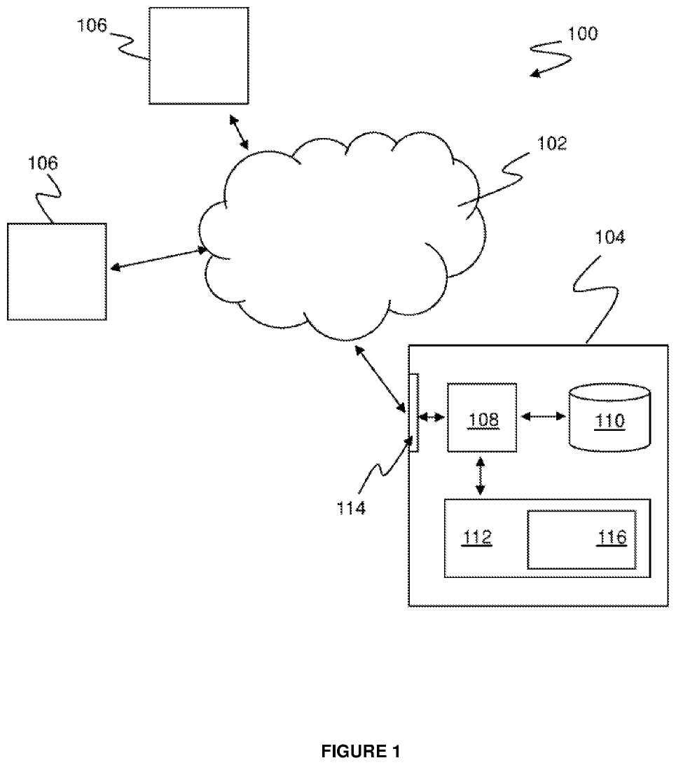

FIG. 1 is a schematic diagram illustrating a system 100 within which embodiments of the present disclosure may be implemented.

The system 100 uses a communications network 102, e.g. the Internet, to facilitate patient/user monitoring utilizing wearable sensor technology and, more particularly, to facilitate a computer-implemented system for automatically generating an emergency response notification for a patient/user.

In the exemplary embodiment 100, a server 104 executes a web server software application for provision of services to user (i.e. patient) devices 106. Communication between the server 104 and the patient devices 106 is thus conveniently based upon standard hypertext transfer protocol (HTTP) and/or secure hypertext transfer protocol (HTTPS).

The patient devices 106 (i.e. ‘clients’) are preferably incorporated into wearable computing devices affixed to the patients, but may also be coupled (via a communications network) to mobile devices such a smart phones, tablets, notebook computers and so forth. As will be appreciated by persons skilled in the communication arts, various mechanisms and technologies are available to provide access to the Internet 102 from mobile devices 106, and all such technologies fall within the scope of the present invention.

The server 104 may generally comprise one or more computers, each of which includes at least one microprocessor 108. The number of computers and processors 108 generally depends upon the required processing capacity of the system, which in turn depends upon the number of concurrent patient devices 106 which the system is designed to support. In order to provide a high-degree of scalability, for example when supporting a global user base, the server 104 may utilise cloud-based computing resources, and/or may comprise multiple server sites located in different geographical regions. The use of a cloud computing platform, and/or multiple server sites, enables physical hardware resources to be allocated dynamically in response to service demand. These and other variations, regarding the server computing resources, will be understood to be within the scope of the present invention, although for simplicity the exemplary embodiments described herein employ only a single server computer 104 with a single microprocessor 108.

The microprocessor 108 is interfaced to, or otherwise operably associated with, a non-volatile memory/storage device 110. The non-volatile storage 110 may be a hard-disk drive, and/or may include solid-state non-volatile memory such as read-only memory (ROM), flash memory, or the like. The microprocessor 108 is also interfaced to volatile storage 112, such as random access memory (RAM), which contains program instructions and transient data relating to the operation of the server 104.

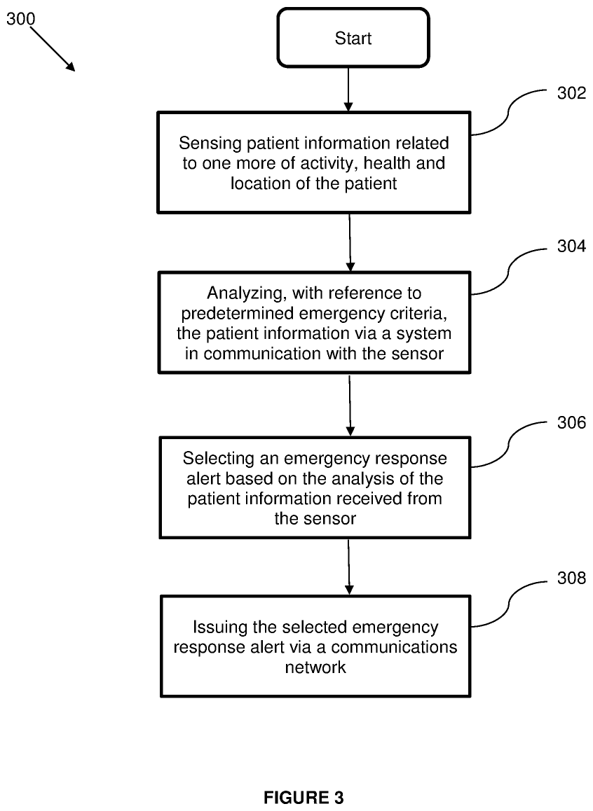

In a conventional configuration, the storage device 110 maintains known program and data content relevant to the normal operation of the server system 104, including operating systems, programs and data, as well as other executable application software necessary to the intended functions of the server 104. In the embodiment shown, the storage device 110 also contains program instructions which, when executed by the processor 108, enable the server computer 104 to perform operations relating to the implementation of services and facilities embodying the present invention, such as are described in greater detail below with reference to FIG. 3 of the drawings. In operation, instructions and data held on the storage device 110 are transferred to volatile memory 112 for execution on demand.

The microprocessor 108 is operably associated with a network interface 114 in a conventional manner. The network interface 114 facilitates access to one or more data communications networks, including the Internet 102, to enable communication between the server 104 and the patient devices 106. In use, the volatile storage 112 includes a corresponding body of 116 of program instructions configured to perform processing and operations embodying features of the present invention, for example as described below with reference to FIG. 3 of the drawings.

For example, the program instructions 116 include instructions embodying a web server application. Data stored in the non-volatile 110 and volatile 112 storage comprises web-based code for presentation and/or execution on user devices 106, such as HTML and/or JavaScript code, for facilitating a web-based implementation of a payment transaction service.

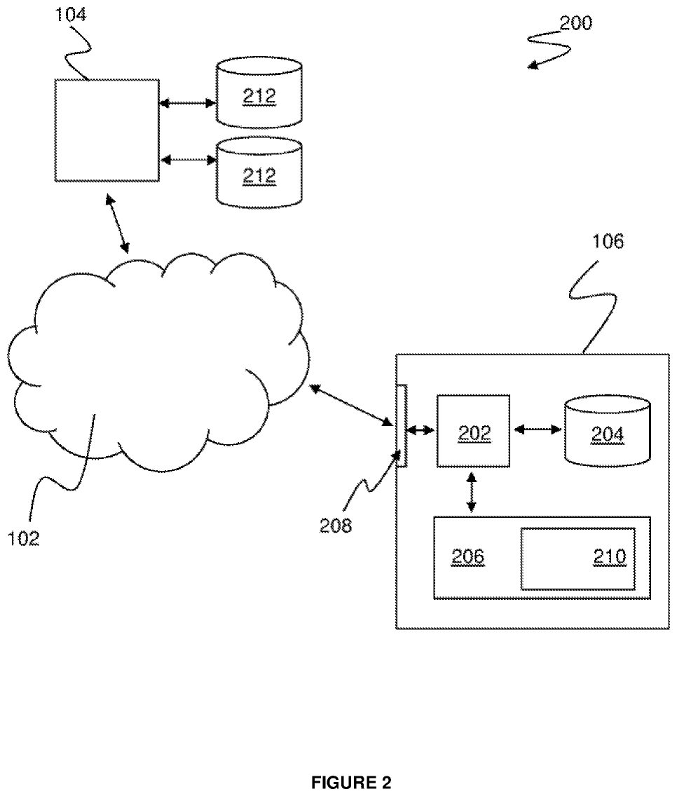

An alternative implementation 200, again by way of example only, is illustrated in the schematic diagram of FIG. 2. In this alternative embodiment, at least a portion of the executable program code implementing the system is executed within the patient devices 106. As shown, each patient device 106 is typically a computing device contained within a wearable device worn by a patient, including at least one microprocessor 202, non-volatile storage 204 and volatile storage 206. Each patient device 106 also has a network interface 208, operably associated with the microprocessor 202 in a conventional manner. Accordingly, the patient devices 106 are able to conduct computational processing by execution of programs stored locally, in the volatile 206 and non-volatile 204 storage, and/or downloaded via the Internet 102 through the network interface 208.

In the embodiment 200 the server 104 may be in communication with one or more databases 212, which may contain patient records relating to patient information for one or more patients, and additionally may include downloadable software components for execution on the patient device 106. For example, a portion of the system may be implemented via program instructions developed in a language such as Java, or some other suitable programming language, which execute on the patient device 106 in order to retrieve data via the server 104, and implement some or all of the functionality of the exemplary system of providing an automated emergency response alert for a patient as described below with reference to FIG. 3.

Client-side implementations may also include downloadable and executable code in the form of browser plugins, such as ActiveX controls for Windows-based browsers, and/or other applets or apps configured for execution within a browser environment or within a smartphone operating system environment, such as an Apple iOS environment or an Android environment.

Various implementations of embodiments of the invention will be apparent to persons skilled in the art of software engineering, including various combinations of server-side and client-side executable program components.

Turning now to FIG. 3, there is shown a flowchart which illustrates an exemplary method 300 of providing an automated emergency response alert for a patient in accordance with the present invention.

A patient will typically be required to wear a patient device 106, which may either directly or indirectly be in connection with a mobile communications device 106 such as, for example, a smart phone, tablets, notebook computers and so forth. In accordance with a representative embodiment of the invention, such a patient device 106 may, for example, be fashioned in the form of a watch, a pendant, a bracelet, a brooch, etc., or any similar form that allows for capture of patient information from the patient. More specifically, the sensor may be positioned against or in close proximity to the patient's skin depending on the type(s) of patient information required. The placement of the device 106 on the patient may range from head, neck, chest, waist, forearm, wrist, or ankle, although alternate placement may also be suggested by a clinician.

The patient device 106 preferably incorporates one or more sensors or MEMS (Micro Electro Mechanical Systems) including, but not limited to, gyroscopes, accelerometers, magnetometers, inclinometers, thermometers, Galvanic Skin Response (GSR) sensors, blood pressure measurement sensors (such as those incorporating the tonometry method and the like), temperature sensors (such as, for example, pyroelectric temperature detectors, resistive temperature detectors, and thermistors), chemical and electrochemical sensors (such as, for example, resistive-type gas sensors, electrochemical gas sensors, colorimetric gas sensors, potentiometric sensors, amperometric sensors, and voltametric sensors), to detect patient and/or environmental information relating to (or being likely to impact) the activity, health, environment and location of the patient wearing the device 106. The patient device 106 may also incorporate one or more infrared receivers, or other types of receivers capable of sensing infrared beams and decoding the data embedded therein. Additionally, the patient device 106 includes a processing unit, memory, and storage as required for a computing device of this type.

The patient device 106, incorporating the computing device, preferably runs operating system software and application software that allows the device 106 to be programmed (either directly or remotely) to perform actions based on certain patient events. In addition, the device 106 may incorporate one or more radio communication means such as, for example, WiFi, Bluetooth, or cellular data modem radios to allow for transmission of data (including patient data) to and from the patient device 106. Alternatively, or in addition, the patient device may incorporate one or more interface ports to allow the device 106 to be programmed, tested, charged, or simply to allow the direct transmission of data (including patient data) into and out of the device 106.

The patient device 106 preferably contains one or more fixed or removable power sources to operate independently of external power. In this regard, the device 106 may incorporate one or more charging ports to charge the internal power source. Alternatively, or in addition, the device 106 may contain one or more induction charging circuits to enable the internal power source to be charged by way of a charging surface such as, for example, a charging mat, charging dongle, and/or charging cradle. In this regard, the device 106 may include one more indicators to inform the patient (or person viewing the device 106) visually or aurally of the state of charge of the internal power source.

The patient device 106 may also contain one or more feedback devices to allow for communication of data or events with the patient. These feedback devices may include lights, vibration motors, visual display units (e.g. LCD screens), and/or speakers. Conversely, the device 106 may include one or more input devices to allow for interaction by the patient (or person interacting with the device 106). These input devices may include microphones, buttons, dials, and/or touch sensors.

Furthermore, the patient device 106 may incorporate various sensing devices (which may, or may not, incorporate MEMS) including capacitance sensors, skin electrodes, pressure sensors, magnetic switches, and/or mechanical switches, to assist in detecting when it is worn by a patient.

In terms of functionality, the patient device 106 is capable of detecting and reporting when the device 106 is being wore, or is not being worn by the patient. Similarly, and as a result of the program instructions uploaded, the patient device 106 make deductions on the activity and posture of the patient such as, for example, whether the patient is sleeping, sitting, reclining, prone, supine, walking, running, shuffling, and/or falling. By interaction with optional beacons (utilising infrared or Bluetooth communication means) placed within a patient's dwelling, the patient device 106 is also able to make deductions as to which room (e.g. bathroom, living room) or area (e.g. garden) where the patient is currently located (by reference to the closest beacon).

The patient device 106 may incorporate one or more internal clocks to provide time base and time reference. As a result, it is possible for the device 106 to aggregate data from each of its sensing devices and timestamp said data, and/or transmit time stamped data to an external system via a communications network. The device 106 is capable of deducing patient activity based on the aggregation of patient data received from its sensing devices, and also capable of storing that patient data (in its raw or aggregated form) onboard the device 106.

In order to ensure connectivity to external communication networks (for the purposes of raising an emergency response alert for the patient), the patient device 106 periodically checks to see if a connection is available to the Internet via one or more of a base station (if one is available to a patient and configured), a mobile device (such as, for example, a smartphone or tablet, if one is configured), a WiFi network (if one is available to a patient and configured), an onboard or outboard cellular data modem (if one is available to a patient and configured). In the event that a base station is configured, the patient device 106 may communicate to it using a low power short range radio frequency protocol such as, for example, Bluetooth, WiFi, ZigBee or XBee. Alternatively, if a smartphone is configured, the patient device 106 may communicate to it using a low power short range radio frequency protocol such as, for example, Bluetooth, WiFi, or NFC. Alternatively, if an outboard cellular data modem is configured, the patient device 106 may communicate to it using a low power short range radio frequency protocol such as, for example, Bluetooth, WiFi, or NFC. Alternatively, if an onboard cellular data modem is configured, the patient device 106 may communicate to it using internal bus protocols such as, for example, i2c, NXP, Serial, or other internal intra-component, intra-circuit board or inter component and inter circuit board protocols.

The patient device 106 may be programmed to periodically communicate via the Internet with an external server 104 in order to transfer and upload patient data to one or more databases 212. As the patient device 106 may be required to issue an emergency response alert for the patient at any time, the device 106 is also programmed to maintain a connection to the Internet at all times, and to immediately seek other means of connectivity in the event that the current means of connectivity is lost or remains unreliable. For example, a patient device 106 may have access to the Internet by means of its connection with a base station operating the Bluetooth protocol. The patient device 106 is programmed to identify when the connection to the base station (and thus the Internet) is lost, and to immediate traverse a hierarchy of alternative connection means in order to re-establish the connection to the Internet.

It may also be preferable that the patient device 106 is waterproof or water resistant in order to allow the device 106 to be used in environments where fluid is likely to come into contact with the device. For example, it would be preferable in the case of an elderly patient that the device 106 is worn at all times including, for example, in the shower where the risk of slippage and/or fall is naturally greater.

At step 302, the method 300 involves sensing, via a sensor (preferably located within the patient device 106), patient information related to one more of activity, health, environment and location of the patient.

The patient information, relative to the patient's activity, may include one or more of the patient's relative movement as a function of time, the patient's movement relative to the patient's predefined movement pattern; and the patient's movement relative to a monitoring hub (e.g. base station) located within the patient's dwelling. The patient's relative movement as a function of time may include, for example, the speed at which the patient is moving, as well as any rapid changes in the height of the sensor worn by the patient (which may be indicative of a fall by the patient). As previously described, and as a result of the program instructions uploaded, the patient device 106 may also make deductions based on the patient information received as to the activity and posture of the patient such as, for example, whether the patient is sleeping, sitting, reclining, prone, supine, walking, running, shuffling, and/or falling. It will be understood by persons skilled in the art how the above described sensors (and particularly MEMS) can be used to obtain this patient information related to the patient's activity.

The patient information, relative to the patient's health, may include one or more of the patient's heart rate, the patient's blood pressure, the patient's body temperature, the patient's blood sugar level; and the patient's electrocardiography. It will be understood by persons skilled in the art how the above described sensors (and particularly MEMS) can be used to obtain this patient information relating to the patient's health.

The patient information, relative to the patient's environment, may include one or more of the ambient temperature of the patient's environment, and the air quality of the patient's environment. It will be understood by persons skilled in the art how the above described sensors can be used to obtain this patient information relation to the patient's environment.

The patient information, relative to the patient's location, may include one or more of the patient's GPS position, and the patient's location relative to a monitoring hub located within the patient's dwelling. It will be understood by persons skilled in the art how the above described sensors can be used to obtain this patient information relating to the patient's location.

Each of the types of patient information is preferably sensed/received by the patient device 106 and either stored to memory on the patient device 106 in the first instance, and/or transmitted to the server 104 via the communications network 102 and uploaded to one or more patient databases 212.

At step 304, the method 300 includes analyzing, with reference to predetermined emergency criteria, the patient information received from the sensor via a system in communication with the sensor.

Prior to use of the patient device 106 by a patient, an optional step may be for the patient to register with the system 100 by accessing a website to create a patient (i.e. patient) profile. As part of this process, the patient (or an authorised person, such as for example a person providing care to the patient) may be required to provide various registration details such as, for example, name, address, contact details, healthcare provider details, username and password. In addition, the patient (or an authorised person, such as for example a person providing care to the patient) may be required to ‘register’ the patient device 106 worn by the patient. Various methods and techniques for device registration will be known to the person skilled in the art and may require, for example, that the patient (or an authorised person, such as for example a person providing care to the patient) input to the website a serial or registration number unique to the specific patient device 106.

More preferably, the pre-use registration of the patient device 106 may also require the patient (or an authorised person, such as for example a person providing care to the patient) to input historical patient information including, but not limited to, pre-existing patient diseases, current medications and diagnosis, as well as patient baseline health vitals including, height, weight, heart rate, blood pressure etc. In a representative embodiment of the present disclosure, all or part of this information may be provided directly by the patient's clinician via a communication link provided between the system 100 and the clinician's computer system as part of either a manual or automated process. In this regard, the clinician may also have the ability to program certain of the predetermined emergency criteria stored within the databases 212 for that patient.

The predetermined emergency criteria, relative to the patient's activity, may include one or more of a predetermined minimum patient movement frequency, a predetermined maximum delay between recorded patient movements, and a predetermined movement pattern for the patient. In an elderly patient, for example, while a high level of movement would not be expected, it would also be irregular if the patient did not exhibit any movement for a prolonged period of time. Similarly, the patient's movement patterns may, for example, specify (based on historical data) that a patient has a certain raised level of activity at particular times throughout the day (e.g. breakfast, lunch, dinner, and scheduled activity periods), and low levels of activity during the night. With regard to elderly patients, whose movements and activity levels tend to be quite routine, these movement patterns can be tracked and recorded to a fairly high level of accuracy (particularly for those elderly patients living in nursing homes).

In relation to the predetermined emergency criteria, relative to the patient's activity, the system 100 may contain certain pre-loaded emergency criteria that would broadly be applicable to the majority of patients. For example, the predetermined emergency criteria, might state for example that during the hours of 8 am and 7 pm the patient should have a minimum movement frequency of two hours. That is, during those hours of the day, the patient should exhibit movement or a moderate nature at least once every two hours.

The predetermined emergency criteria, relative to the patient's health, may include one or more of a predetermined minimum and/or maximum heart rate, a predetermined minimum and/or maximum blood pressure, a predetermined minimum and/or maximum body temperature, a predetermined minimum and/or maximum blood sugar level, and a predetermined range of expected electrocardiography readings. Such predetermined emergency criteria may be set with general parameters (e.g. parameters that would indicate abnormal health conditions for an ‘ordinary’ person) or may be customised based on the patient's own medical history (for example, in the case of a patient with known blood pressure issues, the system would allow for that patient's minimum and/or maximum blood pressure parameters in their predetermined emergency criteria to be modified to reflect that pre-existing condition).

As previously stated, and in a representative embodiment of the present disclosure, the patient's clinician (or responsible carer) may also have the ability to program certain of the predetermined emergency criteria, related to the patient's health, stored within the databases 212 for that patient. For example, in a patient with a pre-existing heart condition or propensity for hypotension (i.e. low blood pressure), the clinician may program as a predetermined emergency criteria for that patient that the patient's blood pressure should not drop below 90/60 for any extended period of time (e.g. greater than 30 seconds). It will be appreciated by the person skilled in the art that the patient's clinician may input a number of predetermined emergency criteria based on their familiarity with the patient's medical history.

The predetermined emergency criteria, relative to the patient's environment, may include one or more of a predetermined maximum ambient temperature, and a predetermined minimum air quality. For example, environmental conditions that could otherwise endanger the health of the patient (such as those caused by fire or heating system malfunction) could be mitigated by setting a maximum ambient temperature of say 50 degrees Celsius. Similarly, an air quality measure could be specified to create an alert when the quality of the air in the patient's immediate environment drops below a specified minimum (as may well occur in the case of a fire or smoky conditions, or where a gas leak has occurred). Such criteria could be manually input or specified, for example, on a country-by-country basis depending on the environmental conditions or specified by an external regulatory body as required.

The predetermined emergency criteria, relative to the patient's location, may include one or more of a predetermined GPS position, and a predetermined maximum distance from the monitoring hub located within the patient's dwelling. It should be appreciated that the predetermined GPS position may also include a ‘geo-fenced’ region defined by GPS coordinates. For example, the ‘geo-fenced’ region may be established to identify the perimeter of a nursing home in which the patient is a resident. As a result, the patient's GPS position relative to the predetermined GPS position (i.e. the ‘geo-fenced’ region) would identify whether the patient is still present within the nursing home.

In relation to the predetermined emergency criteria, relative to the patient's location, the system 100 may contain (based on the registration information provided) basic predetermined location information such as, for example, the patient's ordinary residence or dwelling. As a result, the predetermined emergency criteria for the patient may be pre-loaded and programmed such that the patient should not be more than 100 metres from their ordinary residence or dwelling during certain hours of the day. Alternatively, if the patient has monitoring hub(s) positioned within their ordinary residence or dwelling (e.g. their dwelling or a nursing home in which they reside) the predetermined emergency criteria for the patient may be pre-loaded and programmed such that the patient should not be more than 100 metres from the monitoring hub during certain hours of the day. Alternatively, the patient (or their responsible carer) may input as part of the predetermined emergency criteria a ‘geo-fenced’ region that spans the perimeter of their ordinary residence or dwelling.

At step 306, the method 300 involves selecting an emergency response alert based on the analysis of the patient information received from the sensor. This process includes firstly determining, based on a comparison between the patient information and predetermined emergency criteria, whether an emergency event has occurred, and secondly assigning, based on a comparison between the patient information and predetermined emergency criteria, an alert priority level to the emergency event.

In general terms, at step 306, where the system 300 identifies a discrepancy between the sensed patient information and the predefined emergency criteria, the system 100 deduces that an ‘emergency event’ requiring a response has occurred in respect of the patient. As part of the analysis performed at step 306 of the method, this determination may be based upon a single discrepancy between a specific piece of patient information and its corresponding predetermined emergency criteria. For example, if from the sensed patient information it appears that the patient's pulse has stopped, then this is clearly an emergency event that requires immediate action. Alternatively, as part of the analysis performed at step 306 of the method, this determination may be based upon a cross-analysis of various patient information with respect to the predefined emergency criteria. For example, using the previous example of a patient with a pre-existing heart condition or propensity for hypotension (i.e. low blood pressure), the sensed patient information may indicate that the patient's blood pressure has fallen below the predefined emergency criteria of 90/60. However, before an emergency event can be declared, the system 100 may also take into consideration certain related patient information relevant to the diagnosis. For this specific example, the related patient information may include, but is not limited to, the currently activity of the patient (e.g. resting, sleeping, walking, running), and the patient's heart rate.

While it may be the case that the related patient information (in isolation) does not indicate an emergency event, it may assist with the determination of whether an emergency event has occurred and minimise the occurrence of ‘false positives’. The linking of certain patient information to related patient information can be input by a patient's clinician or responsible carer) in a similar way as previously described. In doing so, it is possible to establish a hierarchy of ‘checks’ within the system 100 when determining whether an ‘emergency event’ has occurred and the type of emergency response that is required.

Similarly, the system 100 may be adapted to build upon a patient's stored event data of emergency events. For example, and again using the same example of a patient with a pre-existing heart condition or propensity for hypotension (i.e. low blood pressure), the system 100 may have stored in the databases 212 event data (including time-based patient information) corresponding to previous emergency events experienced by the patient. As a result, the system 100 is able to apply predictive analytics (and, where necessary, machine learning and artificial intelligence) to the patient information to determine when the same set of patient conditions (that led to the previous emergency event) are beginning to reappear. In this sense, the system 100 attempts to pre-empt when the patient is next likely to suffer an emergency event, and prevent that emergency event from re-occurring. As a result, the system 100 is able to build as a separate predefined emergency criteria a patient emergency profile that is indicative of patient information (over time) that has historically led to an emergency event.

Once the system 100 has determined that an emergency event has occurred to the patient, it is then necessary to assign an alert priority level to the emergency event. The alert priority level may include a low-range emergency response, a mid-range emergency response, or a critical emergency response. In a representative embodiment of the present disclosure, and by way of example, a low-range emergency response may be one that can be adequately addressed by a patient's friends, family, or immediate carers (i.e. the emergency event is not life threatening and does not require an immediate response). Similarly, a mid-range emergency response may be one that the patient's clinician (or in the case of a patient in a nursing home, the nursing or medical staff of the nursing home) can adequately address (i.e. the emergency event is not life threatening and does not require an immediate response). Finally, and also by way of example, a critical emergency response may be one that requires immediate action by emergency medical professionals (such as, for example, an ambulance or emergency services).

As part of this process performed at step 306 of the method, the assignment of the alert priority level may be based upon a single discrepancy between a specific piece of patient information and its corresponding predetermined emergency criteria. For example, if from the sensed patient information it appears that the patient's pulse has stopped, and that an emergency event has occurred, then this is clearly an emergency event that requires immediate action, and one that may be assigned a ‘critical emergency response’ as an alert priority level. Alternatively, as part of the analysis performed at step 306 of the method, the assignment of the alert priority level may be based upon a cross-analysis of various patient information with respect to the predefined emergency criteria. For example, using the previous example of a patient with a pre-existing heart condition or propensity for hypotension (i.e. low blood pressure), the sensed patient information may indicate that the patient's blood pressure has fallen below the predefined emergency criteria of 90/60. The system 100 may deduce that an emergency event has occurred and assign a provisional alert priority level of ‘critical emergency response’. However, before an alert priority level can be assigned, the system 100 may also take into consideration certain related patient information relevant to the diagnosis. For this specific example, the related patient information may include, but is not limited to, the currently activity of the patient (e.g. resting, sleeping, walking, running), and the patient's heart rate. By reference to this related patient information, the system 100 may upgrade or downgrade the provisional alert priority level before assigning the alert priority level. Again, this is beneficial in reducing the occurrence of ‘false positives’, which in the case of a ‘critical emergency response’ would otherwise require the limited resources of emergency response teams to be deployed unnecessarily.

At step 308, the method 300 includes issuing the selected emergency response alert via a communication network. As part of this process performed at step 306 of the method, the step of issuing may further comprise determining, based on the alert priority level assigned to the emergency event, at least one category of recipient for the emergency response alert, and issuing the selected emergency response alert via the communications network to the at least one category of recipient.

The at least one category of recipient may be selected from predefined recipient categories. These predefined recipient categories may comprise primary carer response category, non-critical medical response category, and critical medical response category. As with the categorisation of the alert priority levels, and in a representative embodiment of the present disclosure, the primary carer response category may include members of the patient's friends or family, the non-critical response category may include the patient's clinician (or in the case of a patient in a nursing home, the nursing or medical staff of the nursing home), and the critical medical response category may include emergency medical professionals (such as, for example, an ambulance or emergency services).

As the present invention may be embodied in several forms without departing from the essential characteristics of the invention, it should be understood that the above described embodiments should not be considered to limit the present invention but rather should be construed broadly. Various modifications, improvements and equivalent arrangements will be readily apparent to those skilled in the art, and are intended to be included within the spirit and scope of the invention. The present embodiments are, therefore, to be considered in all respects as illustrative and not restrictive.

1. A computer-implemented method of providing an automated emergency response alert for a patient, the method comprising:
(a) sensing, via a sensor, patient information related to one more of activity, health, environment and location of the patient;
(b) analyzing, with reference to predetermined emergency criteria, the patient information received from the sensor via a system in communication with the sensor;
(c) selecting an emergency response alert based on the analysis of the patient information received from the sensor; and
(d) issuing the selected emergency response alert via a communications network.

2. The computer-implemented method according to claim 1, wherein the patient information, relative to the patient's activity, includes one or more of:
the patient's relative movement as a function of time;
the patient's movement relative to the patient's predefined movement pattern; and
the patient's movement relative to a monitoring hub located within the patient's dwelling.

3. The computer-implemented method according to claim 2, wherein the predetermined emergency criteria, relative to the patient's activity, include one or more of:
a predetermined minimum patient movement frequency;
a predetermined maximum delay between recorded patient movements; and
a predetermined movement pattern for the patient.

4. The computer-implemented method according to claim 1, wherein the patient information, relative to the patient's health, includes one or more of:
the patient's heart rate;
the patient's blood pressure;
the patient's body temperature;
the patient's blood sugar level; and
the patient's electrocardiography.

5. The computer-implemented method according to claim 4, wherein the predetermined emergency criteria, relative to the patient's health, include one or more of:
a predetermined minimum and/or maximum heart rate;
a predetermined minimum and/or maximum blood pressure;
a predetermined minimum and/or maximum body temperature;
a predetermined minimum and/or maximum blood sugar level; and
a predetermined range of expected electrocardiography readings.

6. The computer-implemented method according to claim 1, wherein the patient information, relative to the patient's environment, includes one or more of:
the ambient temperature of the patient's environment; and
the air quality of the patient's environment.

7. The computer-implemented method according to claim 6, wherein the predetermined emergency criteria, relative to the patient's environment, include one or more of:
a predetermined maximum ambient temperature; and
a predetermined minimum air quality.

8. The computer-implemented method according to claim 1, wherein the patient information, relative to the patient's location, includes one or more of:
the patient's GPS position; and
the patient's location relative to a monitoring hub located within the patient's dwelling.

9. The computer-implemented method according to claim 8, wherein the predetermined emergency criteria, relative to the patient's location, include one or more of:
a predetermined GPS position; and
a predetermined maximum distance from the monitoring hub located within the patient's dwelling.

10. The computer-implemented method according to claim 1, wherein in the step of sensing the sensor is positioned against or in close proximity to the patient's skin.

11. The computer-implemented method according to claim 1, wherein the sensor is located within a wearable device worn by the patient.

12. The computer-implemented method according to claim 1, wherein the step of selecting further comprises:
determining, based on a comparison between the patient information and predetermined emergency criteria, whether an emergency event has occurred; and
assigning, based on a comparison between the patient information and predetermined emergency criteria, an alert priority level to the emergency event.

13. The computer-implemented method according to claim 12, wherein the alert priority level comprises one of the following:
a low-range emergency response;
a mid-range emergency response; or
a critical emergency response.

14. The computer-implemented method according to either claim 12, wherein the step of issuing further comprises:
determining, based on the alert priority level assigned to the emergency event, at least one category of recipient for the emergency response alert; and
issuing the selected emergency response alert via the communications network to the at least one category of recipient.

15. The computer-implemented method according to claim 14, wherein the at least one category of recipient is selected from predefined recipient categories.

16. The computer-implemented method according to claim 15, wherein the predefined recipient categories comprise:
primary carer response category;
non-critical medical response category; and
critical medical response category.

17. Software that when installed on a mobile communication device causes the mobile communication device to perform the method of claim 1.

18.-19. (canceled)

20. A computer-implemented system of providing an automated emergency response alert for a patient, the system comprising:
a computer server accessible through a communications network, the computer server arranged to receive patient information through the communications network;
a processor, communicatively coupled to the computer server, to one or more means for graphical display of information, and to one or more means for receiving input, the processor being configured to:
(a) sense, via a sensor, patient information related to one more of activity, health, environment and location of the patient;
(b) analyze, with reference to predetermined emergency criteria, the patient information from the sensor via a system in communication with the sensor;
(c) select an emergency response alert based on the analysis of the patient information received from the sensor; and
(d) issue, via the communications network, the selected emergency response alert via a communications network.

21.-35. (canceled)

36. A mobile communication device comprising:
a sensor, preferably located within a wearable device worn by the patient;
a program memory to store a patient application installed on the mobile communication device;
a data port to facilitate communication with an application server via a communications network; and
a processor to
(a) sense, via the sensor, patient information related to one more of activity, health, environment and location of the patient;
(b) analyze, with reference to predetermined emergency criteria, the patient information from the sensor via a system in communication with the sensor;
(c) select an emergency response alert based on the analysis of the patient information received from the sensor; and
(d) issue, via the communications network, the selected emergency response alert.

37. The mobile communication device of claim 36, further comprising a display device and an input device to facilitate interaction by the patient with the patient application.
